(19) United States
(12) Patent Application Publication
(10) Pub. No.: US 2021/0060804 A1
(43) Pub. Date: Mar. 4, 2021

(54) Hair Clipper with Removable Engageable Blade

(71) Applicant: Manscaped, LLC, San Diego, CA (US)

(72) Inventors: Anh Hao Tran, San Diego, CA (US); Steve King, Solana Beach, CA (US)

(73) Assignee: Manscaped, LLC, San Diego, CA (US)

(21) Appl. No.: 16/963,630

(22) PCT Filed: Nov. 7, 2019
(86) PCT No.: PCT/US2019/060343

§ 371 Date: Jul. 21, 2020

Related U.S. Application Data
(60) Provisional application No. 62/756,671, filed on Nov. 7, 2018.

(51) Int. Cl. B26B 19/38; B26B 19/12; B26B 19/46; A61L 2/10; A61L 2/26

(52) U.S. Cl. CPC B26B 19/3846; B26B 19/12; A61L 2202/11; A61L 2/10; A61L 2/26; B26B 19/46

(57) ABSTRACT

A hair clipper having a battery and motor therein is removably engageable with a cutting blade assembly. The cutting blade assembly is formed of a body with a fixed cutting blade thereon which is in a biased translating engagement with an oscillating blade. An eccentric engaged to the motor powers the oscillating blade to cut hair. A light may be included to illuminate the skin of a user and may be projected in a wavelength to kill pathogens or to encourage skin regrowth.

This application is a U.S. National Phase Application based on International Application Number PCT/US19/60343 filed on Nov. 7, 2019 which claims priority to U.S. Provisional Patent application Ser. No. 62/756671, filed on Nov. 7, 2018, which is incorporated herein in its entirety by this reference thereto.

### FIELD OF THE INVENTION

The present device relates to electric hair clippers. More particularly, the device herein relates to an electric powered hair clipper having a blade assembly positioning both the stationary and moving cutting blades on a removably engageable body thereby allowing complete replacement of the blade assembly with clean or sterile assemblies as needed.

### BACKGROUND OF THE INVENTION

Electric hair clippers have been the primary tool for shaving hair from both humans and animals for decades. Conventionally, such clippers feature a pair of sharpened comb-like blades in close contact, one above the other and the side which slides sideways, relative to each other. An oscillating mechanism, which is electrically powered, oscillates at least one of the blades against the other from side to side. The two blades forming the cutting assembly are conventionally engaged at the distal end of a handle.

In use, such a hair clipper is moved through the hair to be cut so that hair is positioned between the teeth of the comb or stationary blade and is thereafter cut with a scissored action when one blade slides sideways relative to the other. Preferably, the oscillating blade is biased against the comb or stationary blade to enable a scissors like cutting action.

While such hair clippers are widely employed in hair salons, barber shops, and the like, hygiene requires that the cutting assembly be throughly cleaned and disinfected preferably after each use. This is because germs and bacteria from one person can be easily transmitted to a subsequent person on which the hair clipper is employed.

The device herein provides a hair clipper which allows for the complete replacement of the comb and cutting blade with each use. The device herein has a razor with a distal end adapted to operatively engage with the body of the cutting assembly and oscillate one blade while maintaining the comb or stationary blade in a fixed position during use. Once such a use is completed, the blade assembly can be easily removed and replaced with a sterile or otherwise clean blade assembly for each subsequent use. Additionally included is a light projection system which projects one or a plurality of light wavelengths upon the distal end of the blade such that during use the projected light communicates with the distal end of the blade and with the skin of the user. When used for medical hair removal, or for example in a tattoo shop where hair must be removed prior to inking, the device herein allows the user to insert a sterile cutting blade assembly for each use thereby eliminating the need to disinfect.

The forgoing examples of conventional hair clippers and their use are intended to be illustrative and not exclusive, and they do not imply any limitations on the invention described and claimed herein. Various other limitations of the related art are known or will become apparent to those skilled in the art upon a reading and understanding of the specification below and the accompanying drawings.

### SUMMARY OF THE INVENTION

The device herein provides a hair clipper which has a handle with an electric power supply such as a battery which will power an electric motor. The electric motor is engaged to an eccentric rotating shaft, which during use operates to translate a cutting blade against a stationary blade of a cutting assembly.

Both the comb and stationary blade and the oscillating blade of the cutting assembly are operatively engaged on a side surface of a removably engageable body of the cutting assembly. The body is adapted for a frictional or snap-in engagement at the distal end of the handle, in a fashion which operatively engages the oscillating blade to the eccentric rotating shaft.

With the cutting assembly being removably engageable to the handle using the frictional engagement of the body of the cutting assembly on the handle, it is possible for both the stationary blade and the oscillating blade to be concurrently replaced in a single action. This is accomplished by removing a replacement blade assembly from a package and snapping the body portion into a frictional engagement in a recess formed at the distal end of the handle. Should sterile cutting blades be desired or required for each use, the cutting assembly can be sanitized or sterilized and stored in a sealed package which is opened just prior to insertion into the handle.

A unique configuration of the cutting assembly positions the comb or fixed blade in a fixed position on one edge of the body. In the configuration, an oscillating blade is held in biased contact against this fixed blade by a biasing member such as a spring. The spring is configured to concurrently bias and translate the oscillating blade to a centered position upon the fixed comb blade, as it is moved back and forth by the electric motor engaged to an eccentric. Of course, other means to translate the blade can be employed, such as magnetism or other mechanical means, as would occur to those skilled in the art.

The cutting assembly, so configured, can, thus, be replaced in its entirety with each use. This is important where a user may be shaving consecutive customers, such as in a tattoo shop, or in a medical use where hair is shaved from patients. By installing a new, clean, and sterile cutting assembly with each use, germs and pathogens from previous users, which might survive a disinfectant soak in the conventional mode of a trimmer, cannot be transferred since an entirely new cutting assembly is installed each time.

Additionally included may be a light projection system. The light projection system may be configured with a light emitter such as one or a plurality of LEDs or other light emitters. The light emitted by the light emitter is preferably focused with a parabolic or other reflector or internal focusing component to project light in a columnar illumination of the skin of the user, adjacent the distal end of the cutting blade teeth to illuminate it during use.

As such, a light emitter, preferably with a color of between 4500-5500 kelvin has been found to provide the best illumination for users to see the hair and skin during use. In addition to simple illumination the device may include one or a plurality of light emitters which project wavelengths of emitted light which focus to contact both the distal ends of the blade, as well as the skin of a user which the distal end of the blade contacts during use. In addition to illumination of the skin and blade, the different light wavelengths, so projected, may be adapted to kill bacteria and/or encourage new skin cell growth subsequent to use of the hair clipper.

For example, a light emitter generating light between 200 to 400 nanometers (nm), will be highly effective at killing bacteria and viruses by destroying the molecular bonds that hold their DNA together. Especially preferred for killing bacteria and pathogens, is a light emitter projecting light at a wavelength between 260 nanometers and 270 nanometers which has been shown to be particularly effective. Pulsing rather than continuous illumination has shown to enhance results.

Additionally, cell regeneration, scar removal, and smoothing of the skin, can be encouraged where the light emitter emits light at a frequency between 590-760 nanometers. Pulsing of this emitted light has been found especially useful to enhance the results.

Either of these blue or red light frequency spectrums may be employed separately, and also provide illumination, or may be provided in combination by employing multiple light emitters configured for light emission in each of the noted spectrums. With respect to the above description, before explaining at least one preferred embodiment of the herein disclosed hair clipper with removably engageable cutting assembly system invention in detail, it is to be understood that the invention is not limited in its application to the details of construction and to the arrangement of the components in the following description or illustrated in the drawings. The hair clipper invention herein described and shown is capable of other embodiments and of being practiced and carried out in various ways which will be obvious to those skilled in the art. Also, it is to be understood that the phraseology and terminology employed herein are for the purpose of description and should not be regarded as limiting.

As such, those skilled in the art will appreciate that the conception upon which this disclosure is based may readily be utilized as a basis for designing of other hair clipper devices with fully removable cutting assemblies and for carrying out the several purposes of the present disclosed device. It is important, therefore, that the claims be regarded as including such equivalent construction and methodology insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The term “substantially” when employed herein, means plus or minus twenty percent unless otherwise designated in range.

It is an object of the present invention to provide a hand held electric hair trimmer which has an easily removable cutting assembly with both the stationary and translating blade.

It is an additional object of this invention to provide such a hair trimmer where the cutting assembly is engaged to a body portion and adapted for easy engagement and disengagement from the handle in a registered position with an eccentric powering the translating blade.

It is a further object of this invention to provide a hand held electric hair trimmer, which has a light emitter system which may be adapted to illuminate the area being trimmed as well as to kill pathogens on the skin of the user and to encourage new skin cell regrowth subsequent to use of the trimmer.

These and other objects, features, and advantages of the present clipper, as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF DRAWING FIGURES

The accompanying drawings, which are incorporated herein and form a part of the specification, illustrate some, but not the only or exclusive examples of embodiments and/or features of the disclosed hair trimmer. It is intended that the embodiments and figures disclosed herein are to be considered illustrative of the invention herein, rather than limiting in any fashion.

In the drawings:

FIG. 1 depicts the hair clipper device assembled with the body of the cutting assembly engaged to a distal end of a handle in an as-used position in the hand of a user.

FIG. 2 depicts the cutting assembly of the device herein sealed in a sealed container ready for installation on the handle such as in FIG. 5.

FIG. 3 is an enlarged rear view of the operative components of the cutting assembly of the device herein showing the fixed blade engaged to or part of a body and a translating blade biased against the fixed blade and toward a centered position by a biasing member, such as the depicted spring.

FIG. 4 shows the opposite side of the body of the cutting assembly of FIG. 3 showing the fixed blade in the shape of a comb engaged to a first side of the body and prongs for a registered rotating connection to the handle, extending from an opposite side.

FIG. 5 depicts the tilt and snap-in replacement of the cutting assembly to a mount on the handle where, once the prongs are engaged to recesses in the handle, an opening with tapered sides will align with a motor-powered eccentric mounted in a handle cavity, when the cutting assembly is rotated into position as in FIG. 6, and the perimeter edge of the body is frictionally engaged with the handle.

FIG. 6 shows the device as in FIG. 5, where at least one prong projects from one end of the second end of the cutting assembly such as from the body, which is positioned to register the cutting assembly in a proper position with the handle when seated in a mating recess, so that a rotation and pressing of the body will mount the cutting assembly to give the user a new blade.

FIG. 7 shows a mode of the device herein wherein the cutting assembly includes a visual alarm or warning which changes appearance after a time duration or in the presence of pathogens such as bacteria, fungus, viruses, and the like.

FIG. 8 shows a mode of the device herein having a light emitter which preferably employs either a parabolic reflector to gather the light from the focus of the reflector and return it in the depicted concentrated beam or an LED with Total Internal Reflection (TIR) optics built into the light emitter to project a concentrated beam.

FIG. 9 shows a mode of the device herein similar to that of FIG. 8 but showing the device may have a plurality of light emitters where such may emit a concentrated beam as in FIG. 7, of light at a particular frequency which may be employed to kill pathogens and/or encourage cell growth and healing.

### DETAILED DESCRIPTION OF THE INVENTION

In this description, the directional prepositions of up, upwardly, down, downwardly, front, back, top, upper, bottom, lower, left, right and other such terms refer to the device as it is oriented and appears in the drawings and are used for convenience only and such are not intended to be limiting or to imply that the device has to be used or positioned in any particular orientation.

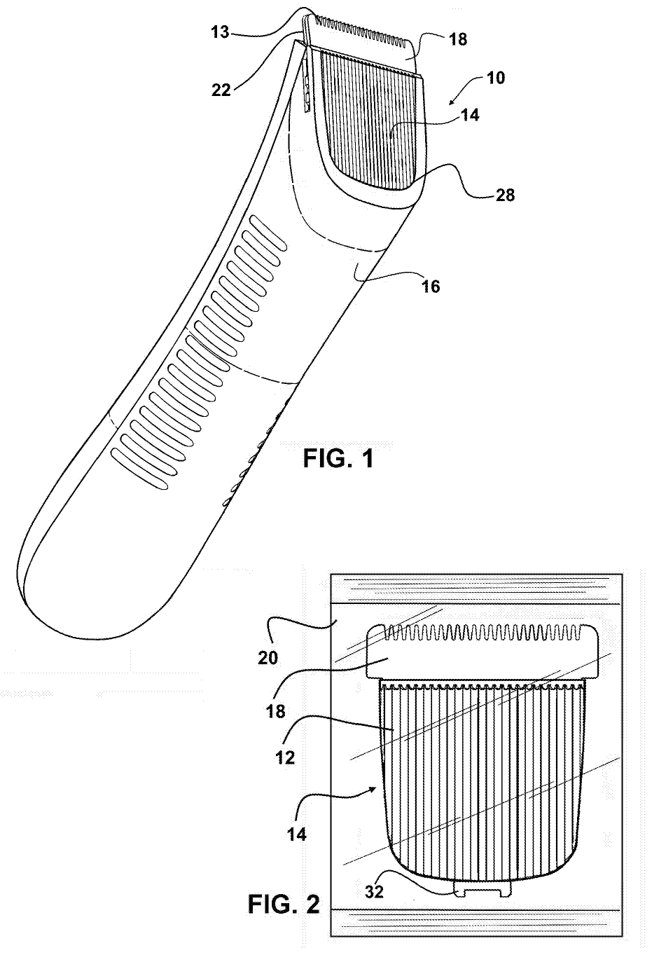

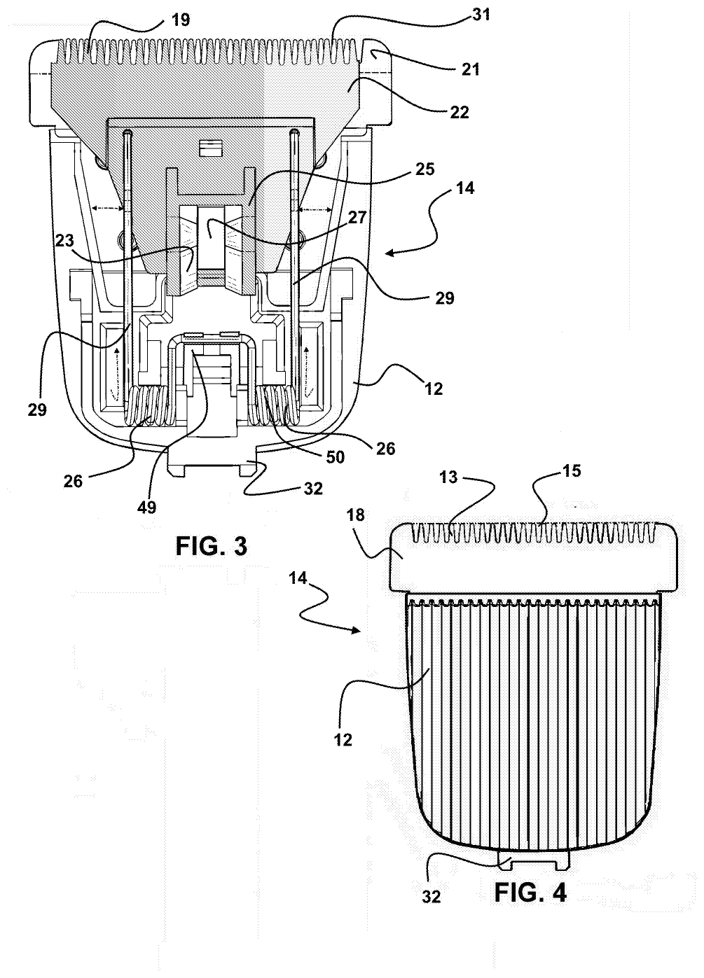

Now referring to drawings in FIGS. 1-9, wherein similar components are identified by like reference numerals, there is seen in FIG. 1, the hair clipper device 10 fully assembled. As shown, a body 12 of the cutting assembly 14 (FIG. 2) is frictionally engaged about a perimeter edge to a recess in the distal end of a handle 16 which has internal cavities housing batteries, wiring, and an electric motor which, while not shown, are well known in the art. A fixed blade 18 is engaged to and extends from a first side of the body 12. This fixed blade 18 portion extends past the distal end surface of the handle 16 when mounted thereon. The fixed blade 18 is slidingly engaged against an oscillating blade 22 of the cutting assembly 14, as shown for example in FIG. 3, by a biasing member such as a spring.

Shown in FIG. 2 is a snap-in replacement cutting assembly 14, having the fixed blade 18 with teeth 13 on a first end of the body 12 and having the oscillating blade 22 slidingly coupled to the cutting assembly 14. The oscillating blade 22 is also biased against a rear side surface of the fixed blade 18. The replacement cutting assembly 14, can be sealed within a package 20 to maintain sterility. Multiple such packaged cutting assemblies 14 can be provided such that the user may easily replace the entire cutting blade assembly 14 by snapping a new one into the handle 16 after each use.

FIG. 3 shows an enlarged rear view of a cutting assembly 14 in assembled form, where the entire cutting assembly 14 is adapted to an easy rotating snap-in engagement with the handle 16. As shown, the cutting assembly 14 has a fixed blade 18 coupled with a first end of the body 12. This fixed blade 18 has a plurality of teeth 13 formed on opposing sides of gaps 15 therebetween (FIG. 4). The fixed blade 18 as shown, has the appearance of a comb coupled with the first end of the body 12. The fixed blade 18 is preferably formed of metal, such as stainless steel, but can be formed of other metals or ceramics.

A translating or oscillating blade 22 is positioned to translate back and forth, in a biased contact, against the rear side surface 21 of the fixed blade 18. This oscillating blade 22 is preferably currently formed of a ceramic material as such has been found to maintain a sharper edge. Although, it may also be formed of metal, such as stainless steel. Further, when formed of ceramic material, it may be impregnated with a pathogen deterrent to kill bacteria and germs and viruses. Such a pathogen deterrent may be one or a combination of deterrents from a group including silver ions, aluminum ions, or copper.

This oscillating blade 22 has a plurality of teeth 19 positioned on opposing sides of gaps 31 in a similar fashion to that of the fixed blade 18.

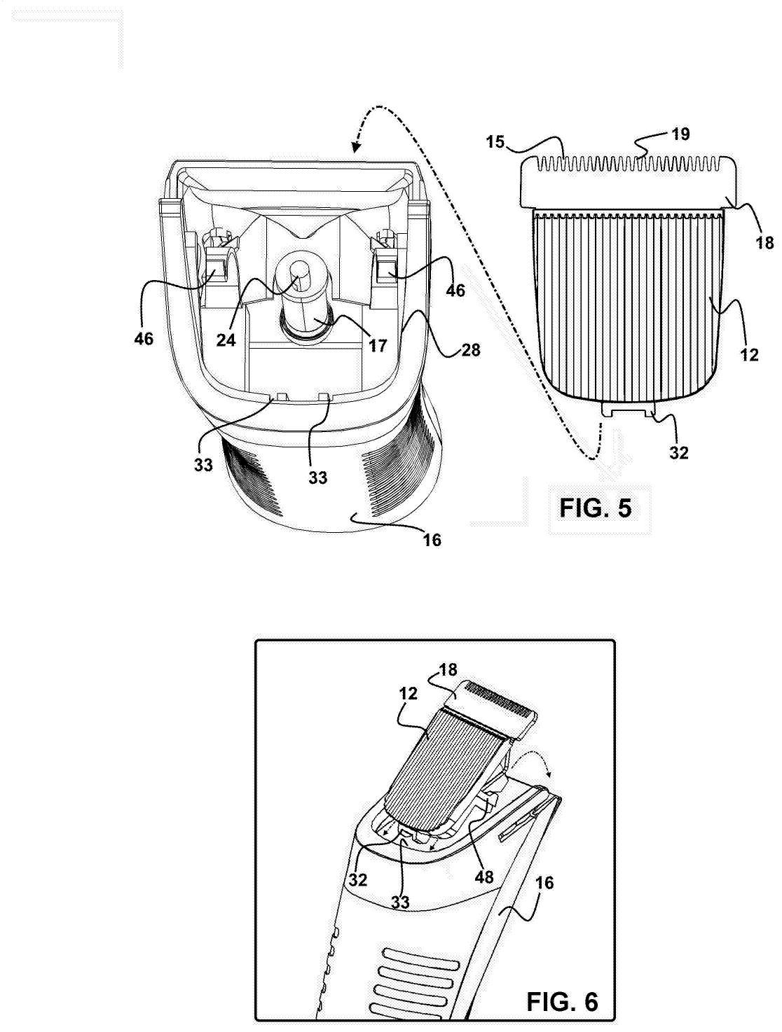

The comb configuration of the section of the oscillating blade 22 having teeth 19, substantially aligns with the teeth 13 and gaps 15 of the fixed blade 18. During use, the oscillating blade 22, in a biased contact against the rear side surface 21 of the fixed blade 18, is translated back and forth by an electric motor having a drive shaft 17 engaged with an eccentric 24 (FIG. 5).

A connector 25 coupled to the oscillating blade 22 has a recess 27 therein, which is configured for operative engagement with the eccentric 24 connected to the drive of the electric motor. This opening 27 on the connector 25 has inward-tapered inner walls 23 which form a funnel of sorts to guide the eccentric 24 into the opening 25 as they slant from a wider opening at a mouth or top end, and into the opening 25 which is sized for width to engage sides of the eccentric 24.

This widened opening mouth, formed by the tapered walls 23, is particularly preferred to render the replaceable blade, formed by the cutting assembly 14, easy to mount when changing it. Such is shown in FIGS. 5 and 5A where at least one prong 32 projecting from the second side of the body 12 of the cutting assembly 14 is positioned to engage with a complimentary shaped slot 33 adjacent an aperture 28 formed on the top end of the handle 16. This aperture 28 is complimentary in a perimeter shape to a perimeter edge of the body 12, such that the body 12 at the perimeter thereof will settle into the aperture 33. The aperture 28 communicates with a cavity in which the eccentric 24 is operatively positioned.

The removable engagement of the prong 32 with the slot 33 forms a hinge which registers the cutting assembly 14 in position to align with and rotate into the aperture 34, wherein biasing connectors 46 temporarily deflect and engage with pins 48 (FIG. 6), to hold the cutting assembly 14 in the mounted position shown in FIG. 1, with the body 12 recessed into the aperture 28.

This formation of a registering temporary hinge by the engagement of one or a plurality of prongs 32 with the slot 33 is especially preferred in that it aligns the perimeter of the body 12 for a recessed fit into the aperture 28, and it concurrently aligns the pins 48, to contact and flex the biasing connectors 46 rearward so they engage over the pins 48 under pressure. Removal is easy by simply pulling on the comb portion of the fixed blade 18, with force to overcome the biased contact of the biased connectors 46 against the pins 48 whereafter the cutting assembly 14 will rotate on the temporary hinge formed by the prong 42 engaged with the slot 33, whereafter it may be lifted free.

Also shown in FIG. 3, is the biasing member is engaged at one end to the body 12 and at an opposite end to the oscillating blade 22. This biasing member such as a spring 26 is configured to impart force to urge or continuously bias the oscillating blade 22 against the rear side surface 21 of the fixed blade 18 during cutting. This biasing force is imparted by arms 29 of the spring, once the spring 26 is engaged with a mount 49 coupled to the body 12 which hold a coiled base 50 of the spring 26 tensioned to bias the arms 29 in a direction toward the body 12.

The spring 26 also employs the arms 29 to impart a centering biasing force, to the oscillating blade 22. A flexing of the elongated arms 29 caused by the eccentric 24 translating the oscillating blade 22, causes the arms 29 to urge the oscillating blade 22 back to a centered position. Thus, both arms 29 when forced from their respective straight or linear configuration, as in FIG. 3, will bias the oscillating blade 22, to which they connect, back to a centered position to urge it in an opposite direction once the motor has moved the oscillating blade 22 off center and a distance to one side or the other of the fixed blade 18. Consequently, the biasing member or spring 26, provides a dual biasing force to the oscillating blade 22 to both urge it against the rear surface 21 of the fixed blade 18 and to move it back to a centered position relative to the body 12.

As noted, the body 12, to which both blades operatively engage, is configured to removably engage within an aperture 28 (FIG. 5) on the handle. During such engagements, a recess 30 engaged with the oscillating blade 22 surrounds the eccentric 24 driven by an electric motor. Contact of the eccentric 24, during rotation of the motor, causes it to force the oscillating blade 22 back and forth in its biased contact with the fixed blade 18 and thereby cut hair which projects through the gaps on the combs formed of teeth and gaps on both the fixed blade 18 and the oscillating blade 22 during use.

In FIG. 4 is shown an opposite side view of the cutting assembly 14 showing a substantially planar body 12 surface. Also shown are the fixed blade 18 engaged to a first side of the body 12. One or a plurality of prongs 32 extend from the opposite or second side of the body 12 from the fixed blade 18 engagement. As noted, the prongs 32 may be employed to engage within a slot 33 in the handle 16 to form a temporary hinge to register and render the cutting assembly 14 easy to mount.

The body 12 is preferably formed of a pliable material such as a polymeric material so it may slightly compress as the perimeter edge of the body 12 engages within the aperture 28 and is surrounded by a ledge or the edge of the aperture 28 formed by the handle 16. This will form a biased frictional engagement of the body 12 and the cutting assembly 14 which is stable for use but easily disengaged by the user pulling upon the fixed blade 18 in a direction away from the aperture 28.

In FIGS. 5-6 is depicted the replacement of the cutting assembly 14 to a biased engagement of the perimeter edge of the body 12 with a wall surrounding the aperture 28 in the handle 16 provided by the configuration herein. During such a rotating engagement, the eccentric 24 connected to the electric motor will align with and settle into position within the recess 30 engaged with the oscillating blade 22. As noted, placing the prongs 32 temporarily engaged with a complimentary shaped slot 33 forms a temporary hinge, which aligns the perimeter of the body 12 with the edge of the aperture 28, and aligns the pins 48 with the biased connectors 46. This allows for an easy rotating snap-in engagement and reverse removal of the cutting assembly.

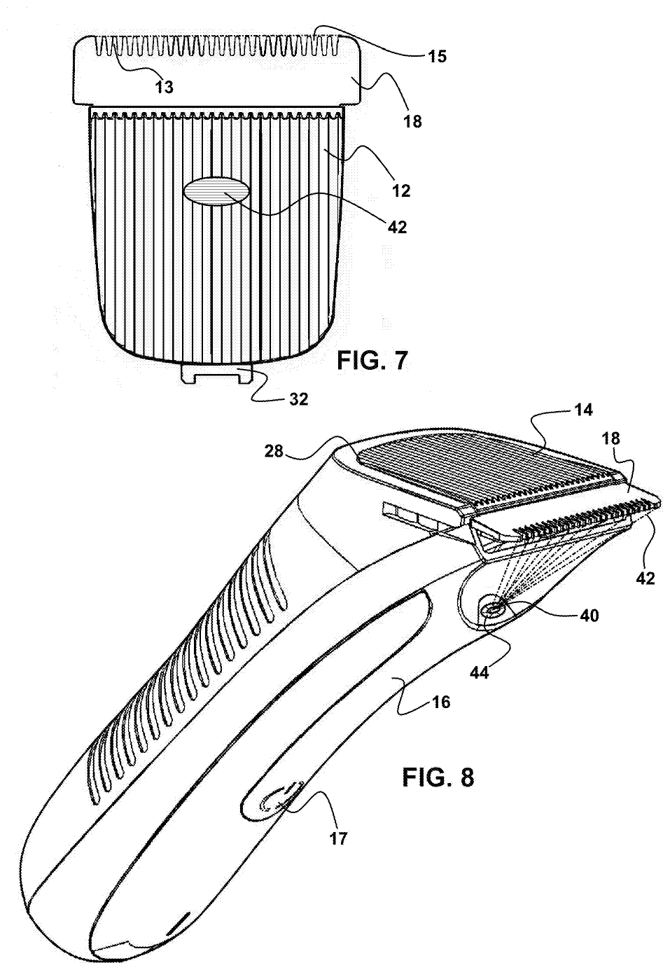

Shown in FIG. 7 is an especially preferred mode of the device herein showing the cutting assembly 14 including a visual signal 42 area or warning which changes visual appearance in the presence of pathogens such as bacteria, fungus, viruses, and the like. The visual signal area 42 is positioned on the front surface of the body 12 of the cutting assembly 14. This visually discernable signal area 42 may also simply be reactive to the air and/or moisture being communicated to the cutting assembly 14 over time, whereupon it changes color or appearance.

In the mode where one or a plurality of pathogens are being monitored, the visual signal area 42 will have material therein which may be paper or polymer or other material treated with appropriate reagents. The reagents are widely available and will change color and thereby visually react to the presence of one or a plurality of pathogens. In this fashion, the user is made aware if the cutting assembly 14 is contaminated with one or a plurality of pathogens to which the visual signal area 42 on the body 12, is configured to visually react.

Alternatively as noted, using an oxygen reactive ink or the like on an insert, if the cutting assembly 14 has been exposed to the atmosphere for a determined period of time, and thus has been used already, the visual signal area 42 can change for example from white, to blue. Such is accomplished using oxygen reactive inks covered by a permeable membrane which is adapted to communicate oxygen to the visual signal area 42 indicate, over a duration to cause the change in color.

Alternatively, if the cutting assembly 14 is provided in a package 20 which blocks light transmission, once the cutting assembly 14 is removed, the visual signal area 42 can include light reactive ink, which will change color to show the user the cutting assembly 14 has already been removed from a package. This is especially useful in medical uses, or for example, for a tattoo artist, where a new and clean blade cutting assembly 14 is required for each patient or client, and if a cutting assembly 14 providing that blade has been removed from the package 20 long enough to change the color of the visual signal area 42, the user is warned the cutting assembly 14 has been exposed and should not be used.

With the visual signal area 42, in any of the above modes, the user, such as those who must have a clean blade provided by the cutting assembly 14 for each use, is provided with a visually discernable signal that the cutting assembly 14 is either fresh and/or uncontaminated, or, has already been used or could have been contaminated by pathogens.

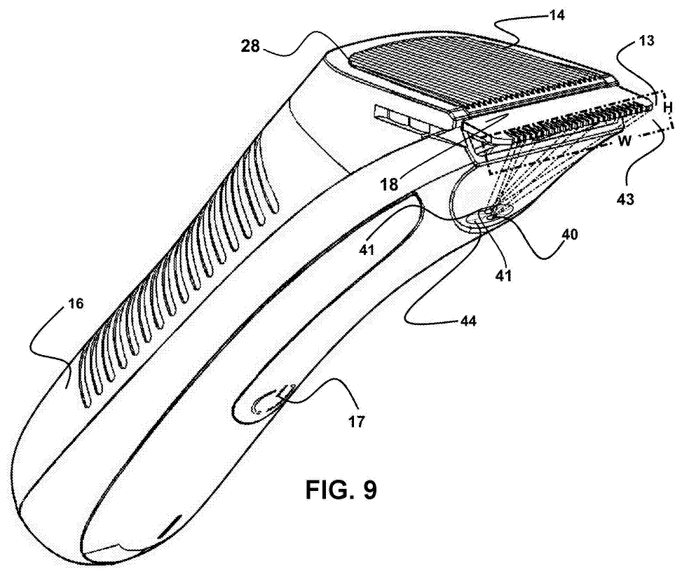

As noted, FIGS. 8-9, depict a mode of the device 10 herein having at least one light emitter 40 which preferably employs a linear focusing component 44 to focus the output illumination from the light emitter 40, to an illumination line 43 which illuminates the teeth 13 when employed, and also the skin of the user adjacent to and encountering the teeth 13, during use and movement of the device 10. As shown, the light emitter 40 is positioned on a side of the handle 16 from which the teeth 13 project and projecting light at an angle toward the teeth 13 when in use. Also shown is the power button 38 which connects an internal battery with the motor powering the eccentric 24 shown in FIG. 5.

By focusing component 44 is meant, a parabolic reflector or other formed light reflector, configured to gather the light emissions from the light emitter 40, and refocuses and/or reflect them, substantially as depicted in FIGS. 8 and 9, preferably in a concentrated beam which forms an illumination line 43 (FIG. 9) when striking the skin or a surface adjacent the distal ends of the teeth 13. Such an illumination line 43 preferably has a width “W” which is equal to or slightly more than a width of the teeth 13 and a height “H” which is ¼ to 1 inches wid, but could be wider if sufficient lumens are projected from the light emitter 40 to light the teeth 13 and adjacent skin thereto effectively. The illumination line 43 may also be focused to be projected in a plane as in FIG. 9, that is within thirty-five degrees of being perpendicular to the line followed by the fixed blade 18.

Another focusing component 44 that is also preferred because of a low electrical current draw, is a light emitter 40 which is an LED configured with Total Internal Reflection (TIR) optics built into the LED light emitter 40. This type of focusing component 44 is internal to the LED and less likely to become dirty and can be made to form the illumination line 43 at the distal ends of the teeth 13 where light will also fall onto the skin of a user during use.

As noted above the light emitter 40 is especially preferred to emit light in a wavelength of 200 to 400 nanometers (nm) because such is highly effective at killing bacteria and viruses by destroying the molecular bonds that hold their DNA together. Especially preferred for killing bacteria and pathogens, is a light emitter projecting light at a wavelength between 260 nanometers and 270 nanometers which experimentation has been shown to be particularly effective. Pulsing of the emitted light from 10-100 percent brightness or lumen emission, rather than continuous illumination has shown to enhance results.

Additionally, cell regeneration, scar removal, and smoothing of the skin can be encouraged where the light emitter emits light at a frequency between 590-760 nanometers. As shown in FIG. 8, the device 10 may have one light emitter 40 which could be at any of the noted wavelengths herein. As shown in FIG. 9, a plurality of light emitters 40 may be used, where each emits light at a respective one of the above wavelengths, to give the user both the ability to kill bacteria and pathogens as well as to encourage new skin growth to smooth the skin.

It should be noted that any of the different depicted and described configurations and components of the clipper herein, can be employed with any other configuration or component shown and described as part of the device herein. Additionally, while the present invention has been described herein with reference to particular embodiments thereof and/or steps in the method of production or use, a latitude of modifications, various changes and substitutions are intended in the foregoing disclosure, and it will be appreciated that in some instance some features, or configurations, of the invention could be employed without a corresponding use of other features without departing from the scope of the invention as set forth in the following claims. All such changes, alternations and modifications as would occur to those skilled in the art are considered to be within the scope of this invention as broadly defined in the appended claims.

Further, the purpose of any abstract included with this specification is to enable the U.S. Patent and Trademark Office, the public generally, and especially the scientists, engineers, and practitioners in the art who are not familiar with patent or legal terms or phraseology, to determine quickly from a cursory inspection the nature and essence of the technical disclosure of the application. Any such abstract is neither intended to define the invention of the application, which is measured by the claims, nor is it intended to be limiting, as to the scope of the invention in any way.

What is claimed is:

1. A hair clipper, comprising:
a handle, said handle having a battery operatively engaged to an electric motor therein;
said handle having a grip on a first end and having an aperture communicating with a handle cavity on a second end thereof;
an eccentric connected to said electric motor, positioned within said handle cavity;
a cutting assembly having a body, said body having a first surface opposite a second surface and having a fixed blade extending from a connection to a first end of said body;
said fixed blade having a plurality of teeth having gaps therebetween;
an oscillating blade, said oscillating blade having a plurality of teeth extending from a first side thereof positioned on opposing sides of gaps therebetween;
a biasing member having a base end thereof coupled with said second side surface of said body;
said biasing member in a connection with said oscillating blade to form a biased contact of said oscillating blade against said fixed blade;
said biased contact holding said teeth and gaps extending from said first side of said oscillating blade in a translating alignment with said teeth and gaps extending from said fixed blade;
said cutting assembly positionable to a removable engagement at said second end of said handle; and
positioning said cutting assembly to said removable engagement coupling said eccentric to an eccentric connector positioned upon said oscillating blade, whereby movement of said eccentric by said motor in contact with said coupling connector, translates said oscillating blade back and forth.

2. The hair clipper of claim 1, additionally comprising:
said biasing member comprising a spring having a coiled portion at said base end and a pair of arms extending from opposing ends of said coiled portion;
distal ends of said arms connected to said oscillating blade on opposite sides thereof; and
said arms concurrently imparting said biased contact of said oscillating blade against said fixed blade, and a centering bias to said oscillating blade to align with a central area of said body, whereby during translation of said oscillating blade by said eccentric, said oscillating blade is biased against said fixed blade and continuously urged toward said central area.

3. The hair clipper of claim 1, additionally comprising:
at least one pin extending from said second side surface of said body;
at least one flexible connector positioned within said handle cavity; and
said pin forming an engagement with said flexible connector to hold said cutting assembly to said removable engagement at said second end of said handle.

4. The hair clipper of claim 2, additionally comprising:
at least one pin extending from said second side surface of said body;
at least one flexible connector positioned within said handle cavity; and
said pin forming an engagement with said flexible connector to hold said cutting assembly to said removable engagement at said second end of said handle.

5. The hair clipper of claim 1, additionally comprising:
at least one tab extending from a second end of said body opposite said first end;
a slot formed at said first end of said handle adjacent a perimeter edge of said aperture;
a recess formed in said eccentric connector, said recess forming said coupling with said eccentric when positioned therein;
said tab removably engageable within said slot to form a hinge;
said cutting assembly rotatable on said hinge to said removable engagement; and
said hinge registering said cutting assembly in a position during rotation of said cutting assembly to said removable engagement which aligns said recess in said eccentric connector.

6. The hair clipper of claim 2, additionally comprising:
at least one tab extending from a second end of said body opposite said first end;
a slot formed at said first end of said handle adjacent a perimeter edge of said aperture;
said eccentric connector located in-between said arms in said connection thereof to said oscillating blade;
a recess formed in said eccentric connector;
said recess forming said coupling with said eccentric when positioned therein;
said tab removably engageable within said slot to form a hinge;
said cutting assembly rotatable on said hinge to said removable engagement; and
said hinge registering said cutting assembly in a position during rotation of said cutting assembly to said removable engagement which aligns said recess in said eccentric connector.

7. The hair clipper of claim 3, additionally comprising:
at least one tab extending from a second end of said body opposite said first end;
a slot formed at said first end of said handle adjacent a perimeter edge of said aperture;
a recess formed in said eccentric connector, said recess forming said coupling with said eccentric when positioned therein;
said tab removably engageable within said slot to form a hinge;
said cutting assembly rotatable on said hinge to said removable engagement; and
said hinge registering said cutting assembly in a position during rotation of said cutting assembly to said removable engagement which aligns said recess for engagement within said eccentric connector and concurrently aligns said pin for engagement with said flexible connector.

8. The hair clipper of claim 4, additionally comprising:
at least one tab extending from a second end of said body opposite said first end;
a slot formed at said first end of said handle adjacent a perimeter edge of said aperture;
a recess formed in said eccentric connector, said recess forming said coupling with said eccentric when positioned therein;
said tab removably engageable within said slot to form a hinge;
said cutting assembly rotatable on said hinge to said removable engagement; and
said hinge registering said cutting assembly in a position during rotation of said cutting assembly to said removable engagement which aligns said recess for engagement within said eccentric connector and concurrently aligns said pin for engagement with said flexible connector.

9. The hair clipper of claim 1, additionally comprising:
a light emitter positioned on said handle;
a focusing component of said light emitter, said focusing component focusing light emitted from said light emitter to form an illumination line illuminating said teeth of said fixed blade and areas of skin of a user adjacent thereto during use of said hair clipper.

10. The hair clipper of claim 9, additionally comprising:
said light emitter emitting light at a wavelength between 200 to 400 nanometers.

11. The hair clipper of claim 9 additionally comprising:
said light emitter emitting light at a wavelength between 260 nanometers and 270 nanometers.

12. The hair clipper of claim 9 additionally comprising:
said light emitter emitting light at a wavelength between 590-760 nanometers as a means to encourage skin cell growth.

13. The hair clipper of claim 12, additionally comprising:
a secondary light emitter, said secondary light emitter emitting light in a secondary wavelength between 260-270 nanometers.
